(19) United States
(12) Patent Application Publication
(10) Pub. No.: US 2005/0147620 A1
(43) Pub. Date: Jul. 7, 2005

(54) Cinnamon formulation for reducing cholesterol and/or glucose levels

(76) Inventor: Karl Bozicevic, Redwood City, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/030,363

(22) Filed: Jan. 5, 2005

Related U.S. Application Data
(60) Provisional application No. 60/534,600, filed on Jan. 5, 2004.
(60) Provisional application No. 60/540,732, filed on Jan. 30, 2004.

(51) Int. Cl. A61K 35/78; A61K 31/455; A61K 31/198; A61K 31/385; A61K 31/225; A61K 31/175; A61K 31/12

(52) U.S. Cl. 424/195.16; 424/739; 514/440; 514/547; 514/592; 514/685; 514/356; 514/565

(57) ABSTRACT

A formulation comprising cinnamon and an active compound such as creatine, a statin drug, niacin, lipoic acid and/or Red Yeast Rice is disclosed. The cinnamon aids in moving the active compound into cells making the active compound more effective as compared to its administration in the absence of cinnamon. Methods of treatment including methods of reducing cholesterol levels, building muscle and treating diabetes are enhanced by the co-administration of cinnamon with another compound.

### CROSS-REFERENCE

This application claims the benefit of U.S. Provisional Application No. 60/534,600, filed Jan. 5, 2004 and 60/540,732 filed Jan. 30, 2004, which applications are incorporated herein by reference.

### FIELD OF THE INVENTION

This invention relates generally to the field of medicine and more particularly to oral drug formulations comprising combinations of compounds, e.g. cinnamon in combination with an active compound.

### BACKGROUND OF THE INVENTION

In order to treat a patient with drugs the patient must be willing and able to take the drugs. Prescription pharmaceuticals can be expensive and even if cost is not a factor many people have an aversion to taking prescription drugs. However, these individuals are often more accepting of the idea of taking non-prescription compounds or formulations. This is in part due to less side effects, lower cost and generally a felling of familiarity with such compounds. Further, the compounds can be purchased without a prescription which avoids the need to see a doctor thereby further reducing expenses and for some patients eliminating the fear of such a visit. If the patient takes the drug the treatment has at least a chance of success. Next the active compound must reach the target in the body.

For any pharmaceutically active compound to be effective it must reach a target site and that site many be inside or on a surface of a cell. Drugs such as insulin bind to cellular receptors and cause the cell membrane to open and/or become permeable to glucose.

Among the many functions performed by peptide and protein hormones in metabolism is the ability to interact with receptors with high specificity. The insulin receptor is present on virtually all cells and at high concentrations on the cells for the liver, skeletal muscles, and adipose tissue. Stimulation of the insulin receptor with insulin is an essential element in carbohydrate metabolism and storage.

Diabetics either lack sufficient endogenous secretion of the insulin hormone (Type I) or have an insulin receptor-mediated signaling pathway that is resistant to endogenous or exogenous insulin (Type II, or non-insulin-dependent diabetes mellitus (NIDDM)). Type II diabetes is the most common form of diabetes, affecting about 5% of individuals in the industrialized nations. In Type II diabetics, major insulin-responsive tissues such as liver, skeletal muscle and fat exhibit the insulin resistance (Haring and Mehnert, Diabetologia 36:176-182 (1993); Haring et al., Diabetologia, 37 Suppl. 2:S149-54 (1994)). The resistance to insulin in Type II diabetes is complex and likely multifactorial but appears to be caused by an impaired signal from the insulin receptor to the glucose transport system and to glycogen synthase. Impairment of the insulin receptor kinase has been implicated in the pathogenesis of this signaling defect. Insulin resistance is also found in many non-diabetic individuals, and may be an underlying etiologic factor in the development of the disease (Reaven, Diabetes, 37:1595-1607 (1988)).

Considerable information is known concerning the insulin receptor itself. The receptor consists of four separate subunits consisting of two identical a and two identical β chains. The β subunits contain a tyrosine kinase activity and the ATP binding sites. The insulin receptor is activated by autophosphorylation of key tyrosine residues in its cytoplasmic tyrosine kinase domain. This autophosphorylation is required for subsequent activity of the insulin receptor. The autophosphorylation stabilizes the activated receptor kinase resulting in a phosphorylation cascade involving intracellular signaling proteins.

At present there are limited pharmacologic approaches to treatment of Type II diabetes. Insulin is currently used as a treatment, but is disadvantageous because insulin must be injected. Although several peptide analogs of insulin have been described, none with a molecular weight below about 5000 daltons retains activity. Some peptides which interact with sites on the β-subunit of the insulin receptor have shown enhancement of the activity of insulin on its receptor (Kole et al., J. Biol. Chem., 271:31619-31626 (1996); Kasuya et al., Biochem. Biophys. Res. Commun., 200:777-83 (1994)). Kohanski and others have reported on a variety of polycationic species that generate a basal effect, but do little to enhance insulin action (Kohanski, J. Biol. Chem., 264:20984-91 (1989); Xu et al., Biochemistry 30:11811-19 (1991). These peptides apparently act on the cytoplasmic kinase domain of the insulin receptor.

In addition, certain non-peptide components have been found to enhance the agonist properties of peptide hormones, but none appear to act directly on the insulin receptor kinase. For instance, the ability of thiazolidinediones, such as pioglitazone, to enhance adipocyte differentiation has been described (Kletzien, et al., Mol. Pharmacol., 41:393 (1992)). These thiazolidinediones represent a class of potential anti-diabetic compounds that enhance the response of target tissues to insulin (Kobayashi, Diabetes, 41:476 (1992)). The thiazolidinediones switch on peroxisome proliferator-activated receptor γ (PPARγ), the nuclear transcription factor involved in adipocyte differentiation (Kliewer et al., J. Biol. Chem., 270:12953 (1995)) and do not have a direct effect on the insulin receptor kinase. Other anti-diabetic agents currently in use include both insulin secretagogues (such as the sulfonylureas) and biguanides (such as metfomin) that inhibit hepatic glucose output.

Bisnaphthalene ureas are known to the literature. They are heavily described as polysulfonic acid derivatives of suramin and as azo dyes. A variety of these polyanionic sulfonic acid derivatives have been established as potential therapeutics for a variety of disease indications. Suramin, described in 1917, is a polysulfonic acid that has been extensively researched (Dressel, J. Chem. Ed., 38:585 (1961); Dressel, J. Chem. Ed., 39:320 (1962)). It has therapeutic uses as an anthehnintic and antiprotozoal. More recently, it has been described as an inhibitor to reverse transcriptase in certain avian and murine retroviruses (De Clercq, Cancer Letter, 8:9 (1979); Mitsuya et al., Science, 226:172 (1984); Gagliardi et al., Cancer Chemother. Pharmacol., 41:117 (1988); Doukas et al., Cancer Res. 55:5161 (1995); Mohan et al., Antiviral Chem., 2:215 (1991)). Large numbers of compounds relating to suramin exist. Most of the suramin analogs which have been reported have multiple sulfonic acid functionality on each aryl ring. Recent studies indicate that polyanionic suramin analogs have anti-angiogenic, antiproliferative activity, and anti-viral activity (Gagliardi et al., Cancer Chemother. Pharmacol., 41:117 (1988); Doukas et al., Cancer Res., 55:5161 (1995); Mohan et al., Antiviral Chem., 2:215 (1991)). A number of bisnaphthylsulfonic acids have been described in the patent literature as complement inhibitors (U.S. Pat. Nos. 4,132,730, 4,129,591, 4,120,891, 4,102,917, 4,051,176). Additionally, there are a number of azo dye patents (DE 19521589, U.S. Pat. No. 3,716,368, DE 2216592, FR 1578556) which disclose polysulfonated naphthalene azo compounds. Bisnaphthalene urea 2-sulfonamide 3-azo compounds have been solely reported as a recording liquid (JP 58191772).

In the last few years there has been considerable interest among athletes in creatine, which occurs abundantly in skeletal muscle. Creatine plays a pivotal role in the regulation and homeostasis of skeletal muscle energy metabolism and it is now generally accepted that the maintenance of phospho-creatine availability is important to the continuation of muscle force production. Creatine may also be involved in other processes concerned with protein synthesis and hypertrophy of muscle fibres during training. Although creatine synthesis occurs in the liver, kidney and pancreas it has been known for sometime that the oral ingestion of creatine will add to the whole body creatine pool, and it has been shown that the ingestion of 20 to 30 g creatine monohydrate (Cr.H.sub.2O) per day for several days can lead to a greater than 20% increase in human skeletal muscle total creatine content. Thus, WO94/02127 discloses the administration of creatine monohydrate in amounts of at least 15 g (or 0.2-0.4 g/kg body weight) per day, for at least 2 days, for increasing muscular strength.

In fact, it was subsequently found that after several days of supplementation (20 g per day) with creatine monohydrate in order to attain initial elevation of the tissue stores, thereafter it takes no more than 2 to 3 g per day to maintain the newly elevated concentration. Supplementation with any bioavailable source of creatine (i.e. creatine supplementation) in an appropriate dose can provide improvements to athletes involved in explosive events, which include all events lasting from a few seconds to a few minutes (such as sprinting, swimming, weight-lifting etc). Endurance performance in events lasting longer than about 30 minutes appear less affected by creatine supplementation except where this involves short periods of increased energy output particularly when the local muscle carbohydrate stores have become depleted. Creatine is a normal food component and is not a drug and its use is not contrary to official regulations. It is possible that the greatest benefits of creatine supplementation are experienced by the elderly, vegetarians or those who eat no meat or fish, since these people tend to have low muscle creatine contents.

Furthermore, creatine and its derivatives have been used in the past but only for the preparation of products with a meaty or savory flavor. For instance, Tonsbeek (U.S. Pat. No. 3,615,600) discloses and is concerned with artificial flavoring, describing mixtures imparting a meaty flavor to foods. Similary de Rooji (U.S. Pat. No. 4,464,409) is concerned with meat flavoring. Yamazaki (JP-A-59035663) prepares a meat flavor by heating a mixture comprising creatine at pH 5.0-7.0 at a temperature of 80-130.degree. C. for 30-120 minutes. Under these conditions most of the creatine is converted to creatinine.

WO 97/45026 discloses an acidic composition for human consumption comprising creatine and its derivatives, the composition being provided as a dry powder or in liquid or semi-liquid form. The compositions disclosed therein are stable at refrigerated temperatures (4.degree. C.) for prolonged periods but stable at ambient temperature for relatively short periods (e.g. up to, but not exceeding, 7 days).

WO 00/74500 discloses compositions comprising creatine and its derivatives suspended in aloe vera gel, which compositions were stable (with respect to the conversion of creatine to creatinine) at room temperature for 2 weeks or more, depending on the initial concentration of creatine in the composition.

Both WO 97/45026 and WO 00/74500 stress the desirability of preventing the conversion of creatine to creatinine, and neither document suggests the deliberate addition of creatinine to a creatine-containing composition intended for human consumption.

It would be a great advantage to present a composition for human consumption, in which the creatine is more readily taken up by cells.

### SUMMARY OF THE INVENTION

A formulation for oral administration to a human is disclosed which is comprised of cinnamon in combination with another component e.g. niacin, Red Yeast Rice and/or creatine which other component has its efficacy enhanced by cinnamon. In addition to creatine the other component may comprise stable and non-hygroscopic creatine salts (e.g., creatine taurinate) and/or a creatine derivative such as a creatine ethyl ester having enhanced nutritional and/or therapeutic efficacy and also relates to the compositions which can be used as energizing dietary supplements, nutraceuticals, health foods and drugs which have their effectiveness enhanced when formulated and administered with cinnamon.

Dietary supplements, nutraceuticals and health foods containing substances of natural origin as active ingredients have become more and more widespread, arousing the interest of ever wider consumers classes.

Creatine is but one of the natural products which, thanks to its physiologic activity, has brought about a major interest both in the scientific community and the consumers.

An aspect of the invention is an oral formulation comprised of therapeutically effective amounts of cinnamon and creatine and/or a creatine ethyl ester.

Another aspect of the invention is an oral formulation comprising a therapeutically effective amount of cinnamon and Red Yeast Rice prepared by fermenting red yeast (Monascus purpureus) with white rice and extracting to substantially remove any citrinin.

Yet another aspect of the invention is an oral formulation comprising cinnamon and lipoic acid.

Still another aspect of the invention is a method of treating a human (specifically to reduce cholesterol and serum glucose levels) by repeatedly administering all or any of the formulations described herein.

Yet another aspect of the invention is an oral formulation comprising cinnamon in combination with a statin drug e.g., a statin drug chosen from any of atorvastatin (Lipitor®), fluvastatin (Lescol®), lovastatin (Mevacor®, Altocor®), pravastatin (Pravachol®), rosuvastatin (Crestor®), and simvastatin (Zocor®).

Another aspect of the invention is an oral formulation comprising cinnamon in combination with niacin, red yeast rice and/or lipoic acid.

Another aspect of the invention is an oral formulation comprising coenzyme Q-10 alone or with cinnamon and niacin and a statin drug chosen from any of atorvastatin (Lipitor®), fluvastatin (Lescol®), lovastatin (Mevacor®, Altocor®), pravastatin (Pravachol®), rosuvastatin (Crestor®), and simvastatin (Zocor®).

These and other objects, advantages, and features of the invention will become apparent to those persons skilled in the art upon reading the details of the formulations and methods as more fully described below.

### DETAILED DESCRIPTION OF THE INVENTION

Before the present formulations and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. The present disclosure controls to the extent it conflicts with any incorporated publication.

It must be noted that as used herein and in the appended claims, the singular forms “a”, “an”, and “the” include plural referents unless the context clearly dictates otherwise. Thus, for example, reference to “a drug” includes a plurality of such drugs and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

### Creatine

Creatine is an organic, nitrogen compound present in considerable amounts in the skeletal muscle tissue of vertebrates wherein about ⅔ thereof occurs as creatine phosphate.

Creatine is biosynthesized mainly in the liver and kidneys from three amino acids: glycine which provides the carbon skeleton, arginine which releases the amidino group and methionine which releases the methyl group. Creatine is excreted with urine as creatinine. Creatine can be taken with the diet since it is principally present in meat. However, in order to take 10 grams/day of creatine, 2.5 kg of meat should be eaten. The exogenous supply and endogenous biosynthesis must compensate for the daily turn-over of creatine to creatinine which in a 70-kg male subject can be estimated at about two grams.

The physiologic role of creatine is extremely important: principally in the skeletal muscle, but in the brain, liver and kidneys as well, creatine—by reversibly taking up ATP's phosphate groups—plays the role of reservoir of the energy-rich phosphate radicals. This reaction is critically important since ATP can not be stored in tissues in excess of a very limited threshold. It is creatine phosphate whose content in tissues is five times as much that of ATP, which provides for phosphate groups supply. Following a moderately wearying physical exertion, the creatine phosphate present in the skeletal muscle decreases in a far relevant amount than ATP does, thus showing that creatine phosphate rephosphorilates ADP as ATP becomes dephospharilated.

When the rate of ATP's metabolic production exceeds ATP's utilization, this results in creatine phosphate formation. Creatine phosphate is, therefore, a reservoir of immediately available energy, suitable for counterbalancing energy demands exceeding ATP's synthesis rate in metabolic phosphorylation processes.

Creatine is mainly taken by athletes and sportsman insofar as it increases the skeletal musculature if its intake is accompanied by lasting physical exertion. Creatine intake results in a lowering of fat while it enhances skeletal muscle. Recent researches have shown that the combined intake of creatine and carbohydrates enhances creatine effects owing to insuline production that is stimulated by simple sugars which likely play a role in creatine exportation to muscle cells.

The term “lipoic acid” is intended to mean .alpha.-lipoic acid which is a chiral molecule also known as thioctic acid; 1,2-diethylene-3 pentanoic acid; 1,2-diethylene-3 valeric acid; and 6,8-thioctic acid. Unless specified the term covers the racemic mixture as well as any other (non-50/50) mixture of the enantiomers including substantially pure forms of either the R-(+) or the S-(−) enantiomer. Further, unless specified otherwise the term covers pharmaceutically acceptable salts (e.g. Na and K salts) and amides, esters and metabolites of the acid. The molecule formula is C₈H₁₄O₂ S₂ the molecular weight is 206.32 and it has a pKa of 4.7. In referring to pharmaceutically acceptable salts the term is intended to encompass a conventional term of pharmaceutically acceptable acid addition salts which refer to salts which retain the biological effectiveness and properties of the free-base form of the acid and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malconic acid, succinic acid, maleic acid, fumaric, tartaric acid, citric acid, benzoic acid, cinnamic acid, mandelic acid, methanesulfonic acid, ethane sulfonic acid, p-toluene sulfonic acid, salicylic acid and the like. The same is true with respect to amides, esters and metabolites that is those forms which can be formed and maintain biological effectiveness and not have significant undesirable biological properties. (See U.S. Pat. No. 6,572,888)

The terms “excipient material” and “Carrier” are used interchangeably here and intended to mean any compound forming a part of the formulation which is intended to act merely as a carrier i.e. not intended to have biological activity itself. The carrier may have an influence on the biological activity of the active compound such as by providing controlled release of that compound. Various non-toxic, biodegradable materials such as polymers, and including all or any of ployethylene glycol, microcrystalline cellulose, hydroxypropylmethyl cellulose, ethylcellulose, cellulose acetate butyrate, cellulose acetate phthalate, polyvinyl acetate phthalate, hydroxypropylmethyl cellulose phthalate, polyethylene oxide, polyvinylpyrrolidone, polyethylene glycol, poly-DL-lactide-co-glycolide, dicalcium phosphate, calcium sulfate, and mixture thereof may be used as carriers.

Niacin refers to vitamin B-3 also known as nicotinic acid. The term is used here to include pharmaceutically acceptable salts of nicotinic acid as well as amides of nicotinic acid and in particular nicotinamide.

The terms “treating”, and “treatment” and the like are used interchangeably herein to generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting it's development; or (c) relieving the disease, i.e. causing regression of the disease and/or it's symptoms or conditions. The invention is directed towards treating patient's suffering from disease related abnormally high levels of either or both of cholesterol and/or glucose and are related to the effects of either or both of these over long periods of time. The present invention is involved in preventing, inhibiting, or relieving adverse effects attributed to high levels of cholesterol and/or serum glucose over long periods of time.

The terms “synergistic”, “synergistic effect” and the like are used interchangeably herein to describe improved treatment effects obtained by combining two or more compounds in formulations of the invention. Although a synergistic effect in some fields means an effect which is more than additive (e.g., one plus one equals three) in the field of treating humans to reduce cholesterol or decrease glucose levels an additive (one plus one equals two) or less than additive (one plus one equals 1.2) effect may be synergistic. For example, if a patient has an abnormally high glucose level, e.g. 400 mg/dl, that patient's glucose level might be reduced to 300 mg/dl by the conventional orally effective antidiabetic compound. Further, at a different time the same patient with a glucose level of 400 mg/dl might be administered a different orally effective antidiabetic compound which compound reduced the patient's glucose levels from 400 to 300 mg/dl. However, if both orally effective antidiabetic compounds are administered to the patient one would not ordinarily expect an additive effect thereby obtaining a reduction to 200 mg/dl and may obtain no more of a reduction in glucose level than when either drug is administered by itself or obtain less of a reduction than either by itself. If additive effects could always be obtained then diabetes could be readily treated in all instances by coadministering several different types of orally effective antidiabetic compounds. However, such has not been found to be an effective treatment. In connection with the present invention coadministration of formulations of the invention comprised of cinnamon with another active compound will provide improved the effects which are synergistic, i.e. greater than the effects obtained by the administration of either composition by itself.

The term “quick release formulation” refers to a conventional oral dosage formulation. Such a formulation may be a tablet, capsule or the like designed to provide for substantially immediate release of the active ingredient and includes enteric coated oral formulation which provide some initial protection to the active ingredient and thereafter allow substantially immediate release of substantially all the active ingredient. A quick release formulation is not formulated in a manner so as to obtain a gradual, slow, or controlled release of the active ingredient.

### Combination Formulations

Cinnamon acts directly on muscle cells to stimulate glucose transport. The effect on serum glucose reduction obtained with cinnamon may be sufficient for some patients. However, if an insufficient glucose lowering effect results the cinnamon may be supplemental with one or more orally effective antidiabetic agents selected from the group consisting of sulfonylureas, biguanides and thiazolidiones.

Useful sulfonylureas include tolbutamide and glipizide and related compounds such as Amaryl, Pandin and Starlix. These drugs target pancreatic beta cells and stimulate these cells to release insulin. The biguanides include compounds such as metformin, phenformin and buformin. These compounds act on the liver to decrease hepatic glucose output and on the intestine to block glucose uptake into the blood. Thiazolidinediones include compounds such troglitazone, rosaglutazone and pioglitazone. These compounds are believed to sensitize muscle and fat cells to insulin.

Although all or any orally effective antidiabetics can be formulated with or administered along with the formulation of the invention it is preferable to administer metformin (particularly metformin Hydrochloride tablets sold as Glucophage.RTM.) with controlled release cinnamon formulations of the invention. Some particularly preferred formulations include 1,000 mg cinnamon and 500 mg of metformin hydrochloride or if a larger dose is needed 2,000 mg of cinnamon and 1,000 mg of metformin hydrochloride. Additional enhanced effects may be obtained by taking cinnamon with vitamin C and/or vitamin E. For example a patient might take 1,000 mg/day of cinnamon 1,000 to 3,000 mg/day of vitamin C and 400 to 800 mg/day of vitamin E.

Examples can be provided by administering to patients coadministration of controlled release cinnamon formulations of the present invention in combination with other treatments conventionally used to lower serum glucose levels. Synergistic effects can obtained, i.e. the combination of cinnamon controlled release formulations of the invention with other therapeutic agents can provide results greater than results which might be expected with the administration of either composition by itself.

### Excipient Material

Examples provided here show that formulations of the invention may comprise different amounts and ratios of active ingredient and excipient material. Further, different excipients can be used. Particularly preferred excipients and amounts used are recited in the Examples. However, upon reading the disclosure those skilled in the art will come to understand the general concepts of the invention and will recognize that other excipients, amounts, ratios and combinations might be used to obtain the results first shown here.

The type and amount of excipient material is added to obtain a formulation with two important characteristics. First, the resulting formulation protects the active ingredient from chemical degradation in the patient's gastrointestinal tract. A formulation of pure, unprotected cinnamon is not part of the scope of the present invention in that pure cinnamon is degraded to some degree in the gastrointestinal tract. Although the formulation need not protect 100% of the cinnamon from degradation to come within the scope of the invention it should protect at least 90% or more, preferably 95% or more and more preferably 99% or more of the cinnamon from degradation. Although multiple doses of an oral formulation could be taken it is preferable to design the dosage such that a single dose is taken at each dosing event—preferably three times a day and more preferably twice a day. The better the active ingredient is protected from degradation the less active ingredient is needed in the original dosage thereby reducing manufacturing costs and increasing profits. The formulation must protect at least as much of the dose as is needed to obtain a pharmacological effect and preferably obtain the desired treatment results, e.g. maintaining a desired serum level needed to obtain a reduced serum glucose level over time.

The second necessary characteristic of the formulation is that it does not release all of the active ingredient at one time but rather releases the active ingredient gradually over time at a controlled rate of release which rate is preferably constant over 4 hours or more. This is particularly important because (1) cinnamon has a relatively short half life and (2) a desired level of cinnamon in blood serum must be maintained over a long period to obtain the desired effect. If all of the cinnamon is released at once it will all enter the circulatory system at once and be metabolized in the liver thereby causing the cinnamon serum level to drop below the desired level. When this occurs the effect on reducing glucose levels is suboptimal.

### Typical Formulations

A typical formulation of the invention will contain about 50% to 70% by weight of cinnamon and a particularly preferred formulation will comprise 60% by weight of cinnamon. Assuming a formulation with 60% by weight of cinnamon with the remaining 40% being excipient material there are a number of possible components which could be used to make up that 40%. A generalized and specific description of such is provided below:

| | | |
|---|---|---|
| (1) | cinnamon | 60% |
| | organic polymer | 40% |
| | TOTAL | 100% |
| (2) | cinnamon | 60% |
| | organic polymer | 34.5% |
| | Inorganics | 5.5% |
| | TOTAL | 100% |
| (3) | cinnamon | 60% |
| | organic polymer | 30%-40% |
| | Inorganics | 10% or less |
| | TOTAL | 100% |
| (4) | cinnamon | 60% |
| | microcrystalline cellulose | 14% |
| | cellulose acetate phthalate aqueous | 15% |
| | dispersion | |
| | ethyl acetate | 2.5% |
| | hydrous magnesium silicate (talc) | 1% |
| | carboxy methyl ether | 4% |
| | magnesium stearate | 0.5% |
| | TOTAL | 100% |
| (5) | cinnamon | 60% |
| | microcrystalline cellulose | 10-30% |
| | cellulose acetate phthalate aqueous | 5-25% |
| | dispersion | |
| | Polyvinylpyraolidone | 1-5% |
| | ethyl acetate | 1-5% |
| | hydrous magnesium silicate (talc) | 0.5-3% |
| | carboxy methyl ether | 1-5% |
| | magnesium stearate | 0.5-1.5% |
| | TOTAL | 100% |
| (6) | cinnamon | 60% |
| | microcrystalline cellulose, NF | 14% |
| | (Avicel PH 101) | |
| | Aquacoat CPD-30 (30% solids w/w) | 15% |
| | Plasdone K29/32, USP | 3% |
| | Carbopol 974P, NF | 2.5% |
| | Talc, USP | 1.0% |
| | croscarmellose sodium, NF (Ac, di-Sol) | 4.0% |
| | Magnesium Stearate, NF | 0.5% |
| | TOTAL | 100% |
| (7) | cinnamon | 60% |
| | microcrystalline cellulose, NF | 10-30% |
| | (Avicel PH 101) | |
| | Aquacoat CPD-30 (30% solids w/w) | 5-25% |
| | Plasdone K29/32, USP | 1-5% |
| | Carbopol 974P, NF | 1-5% |
| | Talc, USP | 0.5-3% |
| | croscarmellose sodium, NF (Ac, di-Sol) | 1-5% |
| | Magnesium Stearate, NF | 0.5-1.5% |
| | TOTAL | 100% |

Those skilled in the art will recognize that there are endless possibilities in terms of formulations and that a margin of error e.g. .+−0.20% or more preferably .+−0.10% should be accounted for with each component. Even if the formulations are limited to the relatively few compounds shown above the formulation could be changed in limitless ways by adjusting the ratios of the components to each other. The important feature of any formulation of the invention is that the cinnamon be released in a controlled manner which makes it possible to maintain therapeutic levels of cinnamon over a substantially longer period of time as compared to a quick release formulation. A particularly preferred formulation will quickly obtain a therapeutic level and thereafter decrease the rate of release to closely match the rate at which cinnamon is being metabolized thereby maintaining a therapeutic level in the patient over a maximum period of time based on the amount of cinnamon in the oral dosage formulation. Some general types of controlled release technology which might be used with the present invention are described below followed by specific preferred formulations.

### Controlled Release Technology

Controlled release within the scope of this invention can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-release Dosage Forms, 1987 (CRC Press, Inc.).

There are corporations with specific expertise in drug delivery technologies including controlled release oral formulations such as Alza corporation and Elan. A search of patents, published patent applications and related publications will provide those skilled in the art reading this disclosure with significant possible controlled release oral formulations. Examples include the formulations disclosed in any of the U.S. Pat. No. 5,637,320 issued Jun. 10, 1997; U.S. Pat. No. 5,505,962 issued Apr. 9, 1996; U.S. Pat. No. 5,641,745 issued Jun. 24, 1997; and U.S. Pat. No. 5,641,515 issued Jun. 24, 1997. Although specific formulations are disclosed here and in these patents the invention is more general than any specific formulation. This includes the discovery that by placing cinnamon in a controlled release formulation which maintains therapeutic levels over substantially longer periods of time as compared to quick release formulations, improved unexpected results are obtained.

The various controlled release technologies cover a very broad spectrum of drug dosage forms. Controlled release technologies include, but are not limited to physical systems and chemical systems.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods, Theory and Applications, 1980 (CRC Press, Inc.).

Controlled release drug delivery systems may also be categorized under their basic technology areas, including, but not limited to, rate-preprogrammed drug delivery systems, activation-modulated drug delivery systems, feedback-regulated drug delivery systems, and site-targeting drug delivery systems.

In rate-preprogrammed drug delivery systems, release of drug molecules from the delivery systems “preprogrammed” at specific rate profiles. This may be accomplished by system design, which controls the molecular diffusion of drug molecules in and/or across the barrier medium within or surrounding the delivery system. Fick's laws of diffusion are often followed.

In activation-modulated drug delivery systems, release of drug molecules from the delivery systems is activated by some physical, chemical or biochemical processes and/or facilitated by the energy supplied externally. The rate of drug release is then controlled by regulating the process applied, or energy input.

In feedback-regulated drug delivery systems, release of drug molecules from the delivery systems may be activated by a triggering event, such as a biochemical substance, in the body. The rate of drug release is then controlled by the concentration of triggering agent detected by a sensor in the feedback regulated mechanism.

In a site-targeting controlled-release drug delivery system, the drug delivery system targets the active molecule to a specific site or target tissue or cell. This may be accomplished, for example, by a conjugate including a site specific targeting moiety that leads the drug delivery system to the vicinity of a target tissue (or cell), a solubilizer that enables the drug delivery system to be transported to and preferentially taken up by a target tissue, and a drug moiety that is covalently bonded to the polymer backbone through a spacer and contains a cleavable group that can be cleaved only by a specific enzyme at the target tissue.

While a preferable mode of controlled release drug delivery will be oral, other modes of delivery of controlled release compositions according to this invention may be used. These include mucosal delivery, nasal delivery, ocular delivery, transdermal delivery, parenteral controlled release delivery, vaginal delivery, and intrauterine delivery.

There are a number of controlled release drug formulations that are developed preferably for oral administration. These include, but are not limited to, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic and anionic drugs. Additional information regarding controlled release drug delivery systems may be found in Yie W. Chien, Novel Drug Delivery Systems, 1992 (Marcel Dekker, Inc.). Further information on controlled release formulations may be found in U.S. Pat. No. 6,191,162.

It is possible to produce formulations which provide multiple release rates, or dual release of a drug from one dosage form. The term bilayer refers to two separate direct compression events that take place during the tableting process. In one embodiment, an immediate release granulate is first compressed, being followed by the addition of a controlled release element which is then compressed onto this initial tablet. This may give rise to the characteristic bilayer seen in the final dosage form.

The controlled release properties may be provided by a combination of hydrophilic polymers. In certain cases, a rapid release of the cinnamon may be desirable in order to facilitate a fast onset of therapeutic affect. Hence one layer of the tablet may be formulated as an immediate release granulate. By contrast, the second layer of the tablet may release the drug in a controlled manner, preferably through the use of hydrophilic polymers. This controlled release may result from a combination of diffusion and erosion through the hydrophilic polymer matrix.

The cinnamon of the invention can be incorporated into any one of the aforementioned controlled released dosage forms, or other conventional dosage forms. The amount of cinnamon contained in each dose can be adjusted, to meet the needs of the individual patient, and the indication. One of skill in the art and reading this disclosure will readily recognize how to adjust the level of cinnamon and the release rates in a controlled release formulation, in order to optimize delivery of cinnamon and its bioavailability.

### Therapeutic Indications

The controlled release cinnamon formulations of the present invention can be used to obtain a wide range of desirable effects. Particularly the formulations of the invention are useful in treating essentially any disease state or symptom which is treatable by long term administration of antioxidants. Further, the invention can be used in the treatment of diseases which involve carbohydrate metabolism and blood glucose disposal which includes various forms of diabetes. Further, the invention is useful in the treatment of various adverse effects on the eyes and skin when the adverse effect are due to high levels of free radicals which can be dissipated by the presence of antioxidants or high levels of serum glucose which can be reduced by stimulating basal glucose transport. Maintaining substantially constant levels of cinnamon provides a long term antioxidant effect which assists in immunomodulation and can result in improved liver and kidney function. Because of the long term antioxidant effect in the circulatory system the present invention has a variety of beneficial effects on the cardiovascular system and in the alleviation of certain liver diseases as well as neurodegenerative diseases. A patient infected with HIV can benefit from the enhanced effect obtained on the immune system.

Because of the very minimal toxicity of cinnamon it can be given to a wide range of patients which have different conditions from mild to serious without fear of adverse effects. Further, the controlled release formulations taught here are even safer than quick release formulations in that serum levels obtained are low compared to quick release formulations. One mild side effect experienced by some patients taking controlled release cinnamon is mild headaches over the first few days. The headaches have not been observed with quick release formulations of cinnamon. Patients treated with vasodilators experience the same mild headaches over the first days of treatment. Thus, controlled release cinnamon formulations of the invention can be used as a vasodilator to treat patients with angina. Controlled release cinnamon can be administered alone or with a conventional vasodilator, e.g. with a nitroglycerin transdermal patch.

The data provided here do not show specific treatments of many of the diseases or symptoms mentioned above. However, the invention is believed to be responsible for obtaining a wide range of beneficial effects particularly when the controlled release formulation is administered to patient's over long periods of time, i.e. weeks, months and years. By maintaining substantially constant therapeutic levels of cinnamon in the blood over very long periods of time a range of desirable physiological results are obtained. Stated differently by continually maintaining the constant therapeutic serum levels of the powerful antioxidant and keeping a patient's blood glucose level within a more desirable range the adverse effects obtained from free radicals and high fluctuating glucose levels are avoided.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Controlled Release Cinnamon

In a first step, cinnamon is screened to a particle size range of 150 to 450 microns. The cinnamon is then added to a Glatt (Ramsey, N.J.) fluid bed granulator. The cinnamon particles become the cores for a coated particle. The cores are coated with a 30% w/w aqueous dispersion of EUDRAGIT.RTM. (NE30 D, methacrylic acid ester) and talc. This yields coated particles with a dried coating weight equal to about 10% of the total weight of the coated particle. The inlet air temperature is kept at a temperature of 25 deg C. After drying, the coated particles are screened using a 40 mesh screen.

The resulting, free-flowing particles are then blended and directly compressed using a tableting press according to the following formula:
cinnamon, coated particles 81%
METHOCEL.RTM. K10010%
(methylcellulose)
Microcrystalline cellulose 5%
Stearic Acid 3%
Micronized silica 0.5%
Magnesium Stearate 0.5%

The resulting tablet is a sustained release formulation.

### Example 2

### Controlled Release Cinnamon and Creatine

Cinnamon and Creatine are mixed in proportions of from 1:3 to 3:1 and the mixture is screened to a particle size range of 150 to 450 microns. The mixture is then added to a Glatt (Ramsey, N.J.) fluid bed granulator. Particles of the mixture become the cores for a coated particle. The cores are coated with a 30% w/w aqueous dispersion of EUDRAGIT.RTM. (NE30 D, methacrylic acid ester) and talc. This yields coated particles with a dried coating weight equal to about 10% of the total weight of the coated particle. The inlet air temperature is kept at a temperature of 25 deg C. After drying, the coated particles are screened using a 40 mesh screen.

The resulting, free-flowing particles are then blended and directly compressed using a tableting press according to the following formula:
coated particles of a mixture of cinnamon and creatine 81%
METHOCEL.RTM. K10010%
(methylcellulose)
Microcrystalline cellulose 5%
Stearic Acid 3%
Micronized silica 0.5%
Magnesium Stearate 0.5%

The resulting tablet is a sustained release formulation.

### Example 3

### Controlled Release Cinnamon and Red Yeast Rice

Cinnamon and Red Yeast Rice are mixed in proportions of from 1:3 to 3:1 and the mixture is screened to a particle size range of 150 to 450 microns. The mixture is then added to a Glatt (Ramsey, N.J.) fluid bed granulator. Particles of the mixture become the cores for a coated particle. EUDRAGIT.RTM. (L/S 100, methacrylic acid ester) is dissolved in isopropyl alcohol to form a 15% w/w solution. Triethyl citrate, talc, and water are additionally added to the solution. Total solids content of the resulting mixture is 9.6% w/w. This yields coated particles with a dried coating weight equal to about 10% of the total weight of the coated particle. The inlet air temperature is kept at a temperature of 25 deg C. After drying, the coated particles are screened using a 40 mesh screen.

The resulting, free-flowing particles are then blended and directly compressed using a tableting press according to the following formula:
coated particles of mixture of cinnamon and Red Yeast Rice 81%
METHOCEL.RTM. K100 5%
(methylcellulose)
Microcrystalline cellulose 5%
Stearic Acid 3%
Micronized silica 0.5%
Magnesium Stearate 0.5%

The resulting tablet is protected from the harsh acid environment of the stomach, and is delivered to the small intestine where it is gradually released.

### Example 4

### Controlled Release Cinnamon and Lipoic Acid

Cinnamon and Lipoic Acid are mixed in proportions of from 1:3 to 3:1 and the mixture is screened to a particle size range of 150 to 450 microns. The mixture is then added to a Glatt (Ramsey, N.J.) fluid bed granulator. Particles of the mixture become the cores for a coated particle. EUDRAGIT.RTM. (L/S 100, methacrylic acid ester) is dissolved in isopropyl alcohol to form a 15% w/w solution. Triethyl citrate, talc, and water are additionally added to the solution. Total solids content of the resulting mixture is 9.6% w/w. This yields coated particles with a dried coating weight equal to about 10% of the total weight of the coated particle. The inlet air temperature is kept at a temperature of 25 deg C. After drying, the coated particles are screened using a 40 mesh screen.

The resulting, free-flowing particles are then blended and directly compressed using a tableting press according to the following formula:
coated particles of the mixture of cinnamon and Lipoic Acid 81%
METHOCEL.RTM. K100 5%
(methylcellulose)
Microcrystalline cellulose 5%
Stearic Acid 3%
Micronized silica 0.5%
Magnesium Stearate 0.5%

The resulting tablet is protected from the harsh acid environment of the stomach, and is delivered to the small intestine where it is gradually released.

### Example 5

### Controlled Release Cinnamon and Biguanide

A preblend of 98% w/w CARBOPOL.RTM. 934 (B.F. Goodrich Chemical, lightly cross-linked acrylic acid allyl sucrose copolymer) and 2% w/w micronized silica is prepared. To this mixture, METHOCEL.RTM. K100, stearic acid, and lactose are added according to the following formula:
mixture of cinnamon and biguanide preblend 70%
CARBOPOL.RTM. 934/silica preblend 10%
METHOCEL.RTM. K10010%
stearic acid 5%
lactose 5%

The resulting mixture is tableted using a direct compression tableting press to form a bioadhesive formulation.

### Example 6

### Controlled Release Cinnamond and Atorvastatin (Lipitor®)

A preblend is formed of 98% w/w /cinnamon mixed with atorvastatin combined with 2% w/w CAB-O-SIL.RTM. micronized silica. To this mixture is added guar gum (AQUALON.RTM. G-3), polyvinylpyraolidone (PVP), calcium carbonate, stearic acid, lactose, and magnesium stearate in the following amounts:
Mixture of cinnamon and atorvastatin (Lipitor®)/CAB-O-SIL.RTM. blend 49.5%
guar gum (AQUALON.RTM. G-3)30%
polyvinylpyraolidone (PVP) 5%
calcium carbonate 5%
stearic acid 5%
lactose 5%
magnesium stearate 0.5%

The resulting mixture is tableted using a direct compression tableting press to form a sustained release caplet formulation.

| Item | | | Theoretical | Unit of |
|---|---|---|---|---|
| No. | Item Description | Percent | Quantity | Measure |
| 1 | Mixture of cinnamon | 60 | 4800.0 | g |
| | and atorvastatin | | | |
| | (Lipitor ®) | | | |
| 2 | Microcrystalline | 18 | 1440.0 | g |
| | Cellulose NF (Avicel | | | |
| | PH 101) | | | |
| 3 | Aquacoat CPD (30% | 15* | 4000.0* | g |
| | w/w) | | | |
| 4 | Povidone K29/32, USP | 3 | 240.0 | g |
| 5 | Carbopol 974P | 2.5 | 200.0 | g |
| 6 | Talc, USP | 1 | 80.0 | g |
| 7 | Magnesium Stearate, | 0.5 | 40.0 | g |
| | NF | | | |
| 8 | Purified Water, USP | | | g |
| TOTAL | | 100 | 8000.0 | g |

*Quantity indicates amount of dispersion to be used in granulating. Actual Solids Content - 1200 g - 15% is based on solids content

Before formulating a check should be made of the room and equipment in order to verify that the cleaning procedure has been performed and approved. Weigh and charge cinnamon (Item 1) and Avicel PH 101, (Item 2) in a Hobart Mixer and mix for two (2) minutes with the mixer speed set at 1 or 2. Granulate the Step 2 material by slowly adding Aquacoat CPD (Item 3) until granules are formed. Add additional Purified Water, USP (Item 8) if required, and mix until the granules are formed. Mixer Speed Setting remains at 1-2. Spread the granulation evenly from Step 3 on paper-lined trays and load them into the oven. Dry at 40.degree. C.+−0.5.degree. C. for two (2) hours. Check LOD and record moisture content. If LOD is more than 2%, continue drying until LOD is below 2%. Pass the dried material from Step 5 through a size 14 mesh screen, hand held or using a Quadro Comil. Charge the Step 6 granulation into a V-blender. Charge the Step 7 blend in blender with Povidone K29/32, USP (Item 4) and Carbopol 974P (Item 5) and mix for five (5) minutes. Charge the V-blender with Talc (Item 6) and Magnesium Stearate, NF (Item 7) and blend for three (3) minutes. Empty the blend from the V-blender into a properly labeled tared PE-lined container and record the weights in Step 11. Theoretical weight of blend: 8000.0 g. Lower Limit 95% and Upper Limit 102%. Any discrepancy from these established limits must be reported to Production and Quality Assurance. Any discrepancy must be appropriately investigated and documented. Hold the blend in the in-process Q.C. Hold area for further processing. Using the amounts shown above will result in sufficient formulations to produce above 16,000 300 mg tablets.

### Example 7

### Controlled Release Cinnamon and Fluvastatin (Lescol®)

A preblend is formed of 98% w/w mixture of cinnamon and fluvastatin (Lescol®) combined with CARBOPOL.RTM. 934 (B.F. Goodrich Chemical, lightly cross-linked acrylic acid allyl sucrose copolymer) and 2% w/w micronized silica. To this mixture, cinnamon, METHOCEL.RTM. K100, stearic acid, and lactose are added according to the following formula:
mixture of cinnamon and fluvastatin (Lescolg) preblend 70%
CARBOPOL.RTM. 934/silica preblend 10%
METHOCEL.RTM. KI0010%
stearic acid 5%
lactose 5%

The resulting mixture is tableted using a direct compression tableting press to form a bioadhesive formulation.

### Example 8

### Controlled Release Cinnamon and Lovastatin (Mevacor®, Altocor®)

A preblend is formed of 98% w/w mixture of cinnamon and lovastatin combined with CARBOPOL.RTM. 934 (B.F. Goodrich Chemical, lightly cross-linked acrylic acid allyl sucrose copolymer) and 2% w/w micronized silica. To this mixture, cinnamon, METHOCEL.RTM. K100, stearic acid, and lactose are added according to the following formula:
mixture of cinnamon and lovastatin (Mevacor®, Altocor®) 70%
CARBOPOL.RTM. 934/silica preblend 10%
METHOCEL.RTM. K10010%
stearic acid 5%
lactose 5%

The resulting mixture is tableted using a direct compression tableting press to form a bioadhesive formulation.

### Example 9

### Controlled Release Cinnamon and Pravastatin (Pravachol®)

A preblend is formed of 98% w/w mixture of cinnamon and pravastatin combined with CARBOPOL.RTM. 934 (B.F. Goodrich Chemical, lightly cross-linked acrylic acid allyl sucrose copolymer) and 2% w/w micronized silica. To this mixture, cinnamon, METHOCEL.RTM. K100, stearic acid, and lactose are added according to the following formula:
mixture of cinnamon and pravastatin (Pravachol®) preblend 70%
CARBOPOL.RTM. 934/silica preblend 10%
METHOCEL.RTM. K10010%
stearic acid 5%
lactose 5%

The resulting mixture is tableted using a direct compression tableting press to form a bioadhesive formulation.

### Example 10

### Controlled Release Cinnamon and Rosuvastatin (Crestor®)

A preblend is formed of 98% w/w mixture of cinnamon and rosuvastatin combined with CARBOPOL.RTM. 934 (B.F. Goodrich Chemical, lightly cross-linked acrylic acid allyl sucrose copolymer) and 2% w/w micronized silica. To this mixture, cinnamon, METHOCEL.RTM. K100, stearic acid, and lactose are added according to the following formula:
mixture of cinnamon and rosuvastatin (Crestor®) preblend 70%
CARBOPOL.RTM. 934/silica preblend 10%
METHOCEL.RTM. K10010%
stearic acid 5%
lactose 5%

The resulting mixture is tableted using a direct compression tableting press to form a bioadhesive formulation.

### Example 11

### Controlled Release Cinnamon and Simvastatin (Zocor®)

A preblend is formed of 98% w/w mixture of cinnamon and simvastatin combined with CARBOPOL.RTM. 934 (B.F. Goodrich Chemical, lightly cross-linked acrylic acid allyl sucrose copolymer) and 2% w/w micronized silica. To this mixture, cinnamon, METHOCEL.RTM. K100, stearic acid, and lactose are added according to the following formula:
mixture of cinnamon and simvastatin (Zocor®) preblend 70%
CARBOPOL.RTM. 934/silica preblend 10%
METHOCEL.RTM. K10010%
stearic acid 5%
lactose 5%

The resulting mixture is tableted using a direct compression tableting press to form a bioadhesive formulation.

FORMULATIONS

| | | |
|---|---|---|
| (1) | cinnamon | 150 mg |
| | Red Yeast Rice | 150 mg |
| | carrier | remainder |
| (2) | cinnamon | 100 mg |
| | Red Yeast Rice | 100 mg |
| | Niacin | 100 mg |
| | carrier | remainder |
| (3) | cinnamon | 100 mg |
| | Red Yeast Rice | 100 mg |
| | Niacin | 100 mg |
| | lipoic acid | 100 mg |
| | carrier | remainder |

In any of the formulations the carrier may be any non-toxic, biodegradable material such as polymers, and including all or any of ployethylene glycol, microcrystalline cellulose, hydroxypropylmethyl cellulose, ethylcellulose, cellulose acetate butyrate, cellulose acetate phthalate, polyvinyl acetate phthalate, hydroxypropylmethyl cellulose phthalate, polyethylene oxide, polyvinylpyrrolidone, polyethylene glycol, poly-DL-lactide-co-glycolide, dicalcium phosphate, calcium sulfate, and mixtures thereof.

Further, tablets, capsules and the like may be designed wherein any of the components are present in different amounts, e.g. cinnamon 25-500 mg, Niacin, 25-500 mg, lipoic acid 25-500 mg, Red Yeast Rice, 25-500 mg.

The invention further includes combining cinnamon in amounts of 25-500 mg with other spices such as curcumin in amounts of 25-500 mg.

Various non-toxic, biodegradable materials such as polymers, and including all or any of ployethylene glycol, microcrystalline cellulose, hydroxypropylmethyl cellulose, ethylcellulose, cellulose acetate butyrate, cellulose acetate phthalate, polyvinyl acetate phthalate, hydroxypropylmethyl cellulose phthalate, polyethylene oxide, polyvinylpyrrolidone, polyethylene glycol, poly-DL-lactide-co-glycolide, dicalcium phosphate, calcium sulfate, and mixture thereof may be used as carriers.

| | | |
|---|---|---|
| (1) | cinnamon | 150 mg |
| | Red Yeast Rice | 150 mg |
| | carrier | remainder |
| (2) | cinnamon | 100 mg |
| | Red Yeast Rice | 100 mg |
| | Niacin | 100 mg |
| | carrier | remainder |
| (3) | cinnamon | 100 mg |
| | Red Yeast Rice | 100 mg |
| | Niacin | 100 mg |

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An oral formulation comprising:
cinnamon; and
an additional ingredient chosen from niacin, Red Yeast Rice, creatine, creatine ethyl ester, lipoic acid, curcumin and a sulfonylurea.

2. The formulation of claim 1, further comprising:
a pharmaceutically acceptable carrier.

3. The formulation of claim 1, wherein the formulation is in a form chosen from a tablet, a capsule, a caplet, a powder and a liquid.

4. The formulation of claim 1 in a form chosen from a tablet and a capsule comprise cinnamon in an amount in a range of from about 50 mg to 3000 mg and the additional ingredient comprises niacin and Red Yeast Rice and each are present in an amount of from about 50 mg to about 2000 mg.

5. The formulation of claim 4 comprising 500 mg±20% of cinnamon and 2000 mg±20 of the additional ingredient and further wherein the formulation is in a form of a controlled release tablet further comprising a pharmaceutically acceptable carrier.

6. An oral formulation, comprising:
a combination of cinnamon, niacin, and Red Yeast Rice; and
a pharmaceutically acceptable carrier.

7. The formulation of claim 6, wherein each of the cinnamon, niacin, and Red Yeast Rice is present in an amount in a range for from about 50 mg to about 300 mg±20%.

8. A method of treatment, comprising:
orally administering to a patient an oral formulation comprising therapeutically effective amount of cinnamon in combination with a pharmaceutically active drug.

9. The method of claim 8, wherein the cinnamon is present in an amount in a range of from about 50 mg to about 2000 mg.

10. The method of claim 9, wherein the cinnamon and drug are in a controlled release formulation and released in a manner so as to maintain therapeutical drug levels in the patient for 50% or more longer as compared to a quick release oral formulation.

11. A method of reducing cholesterol, comprising:
administering to a patient an oral formulation comprising a combination of cinnamon and an additional ingredient chosen from niacin, Red Yeast Rice, and lipoic acid.

12. The method of claim 11, wherein the cinnamon and additional ingredient are administered in a combined oral dosage unit.

13. The method of claim 12, wherein the combined oral dosage unit comprises one or more tablets.

14. A method of formulating a tablet, comprising the steps of:
mixing together a pharmaceutically acceptable carrier, cinnamon; and
an additional ingredient chosen from niacin, Red Yeast Rice, and lipoic acid to form a uniform mixture; and
compressing the mixture to form a tablet.

15. A method of treating, comprising:
orally administering to a human patient a therapeutically effective amount of a formulation comprising cinnamon in combination with an ingredient chosen from creatine, creatine ethyl ester, niacin, Red Yeast Rice, and lipoic acid;
repeating the administration of the formulation on at least a daily basis over a period of time.

16. The method of claim 15, wherein the cinnamon is present in an amount of 200 mg to about 2 grams, the creatine is an amount of 300 mg to 3 grams, the Red Yeast Rice in amount of 200 mg to 2 grams and the niacin in an amount of 50 mg to 300 mg.

17. The method of claim 16, wherein the formulations is administered once a day or more for a period of seven days or more.

18. The method of claim 17, wherein the formulation is administered once a day or more for a period of twenty eight days or more.

19. An oral formulation, comprising:
cinnamon; and
a statin drug.

20. The formulation of claim 19, wherein the statin drug is chosen from atorvastatin (Lipitor®), fluvastatin (Lescol®), lovastatin (Mevacor®, Altocor®), pravastatin (Pravachol®), rosuvastatin (Crestor®), and simvastatin (Zocor®).

21. The formulation of claim 20, further comprising:
coenzyme Q-10.

22. An oral formulation, comprising:
a statin drug; and
coenzyme Q-10.

23. The formulation of claim 22, further comprising:
cinnamon.
